(19) United States
(12) Patent Application Publication
(10) Pub. No.: US 2023/0277779 A1
(43) Pub. Date: Sep. 7, 2023

(54) SYRINGES AND INJECTORS WITH CAPACITIVE SENSING AND METHODS OF MAKING AND USING SAME

(71) Applicant: Merck Sharp & Dohme LLC

(72) Inventors: Douglas E. Weitzel, Hamilton, NJ (US); Steven Carl Persak, Morris Plains, NJ (US); Jeffrey C. Givand, Westfield, NJ (US); Christopher Granelli, Chatham, NJ (US)

(73) Assignee: Merck Sharp & Dohme LLC, (omitted)

(21) Appl. No.: 18/317,377

(22) Filed: May 15, 2023

Related U.S. Application Data
(63) Continuation of application No. PCT/US2021/063176, filed on Dec. 14, 2021.
(60) Provisional application No. 63/126,197, filed on Dec. 16, 2020.

(51) Int. Cl. A61M 5/32

(52) U.S. Cl. CPC A61M 5/3287; A61M 5/322; A61M 2205/13; A61M 2205/3317

(57) ABSTRACT

An injecting device includes a reservoir for containing a medicament, a needle in communication with the reservoir and configured to deliver the medicament to a patient’s body, at least two electrodes spaced apart from one another and disposed on opposing sides of the needle, and a capacitance-to-digital converter circuit configured to generate a signal and measure capacitance between the at least two electrodes.

### FIELD OF THE DISCLOSURE

The present disclosure relates generally to syringes and injectors having capacitive sensing capabilities. More specifically, the present disclosure relates to syringes and injectors having capacitive sensing used for assessing insertion of a needle into a patient’s body.

### BACKGROUND OF THE DISCLOSURE

A pre-filled syringe typically includes a glass barrel containing a pharmaceutical product, which is sealed by a stopper. One concern when using pre-filled syringes is known as “dose splitting,” in which the contents of, typically, a prefilled syringe designed for subcutaneous injection is transferred into another container, such as a vial or intravenous bag, in preparation for off-label or otherwise unintended use. Behavior such as “dose splitting” is undesirable, potentially unsafe due to incorrect treatment, and may undermine data collection, for example, for clinical trials. Conventional devices and methods do not provide sterile and accurate techniques for assessing patient compliance.

Thus, there exists a need for devices that improve upon and advance the methods of safely using injectors and syringes, such as pre-filled syringes.

### SUMMARY OF THE DISCLOSURE

In one embodiment, an injecting device includes a reservoir for containing a medicament, a needle in communication with the reservoir and configured to deliver the medicament to a patient’s body, at least two electrodes spaced apart from one another and disposed on opposing sides of the needle, and a capacitance-to-digital converter circuit configured to generate a signal and measure capacitance between the at least two electrodes.

### BRIEF DESCRIPTION OF THE DISCLOSURE

The patent or application file contains at least one drawing executed in color. Copies of this patent or patent application publication with color drawing(s) will be provided by the Office upon request and payment of the necessary fee.

Various embodiments of the presently disclosed syringes and sensors are disclosed herein with reference to the drawings, wherein:

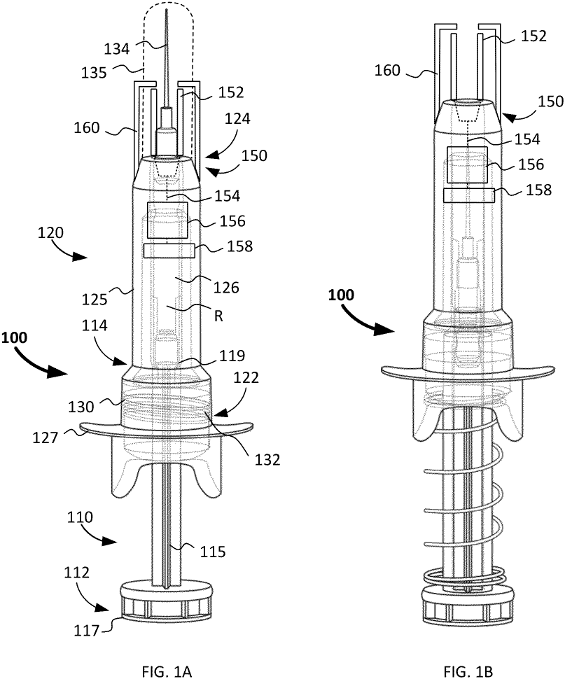

FIGS. 1A-B are schematic front views of a pre-filled syringe having a capacitive sensor;

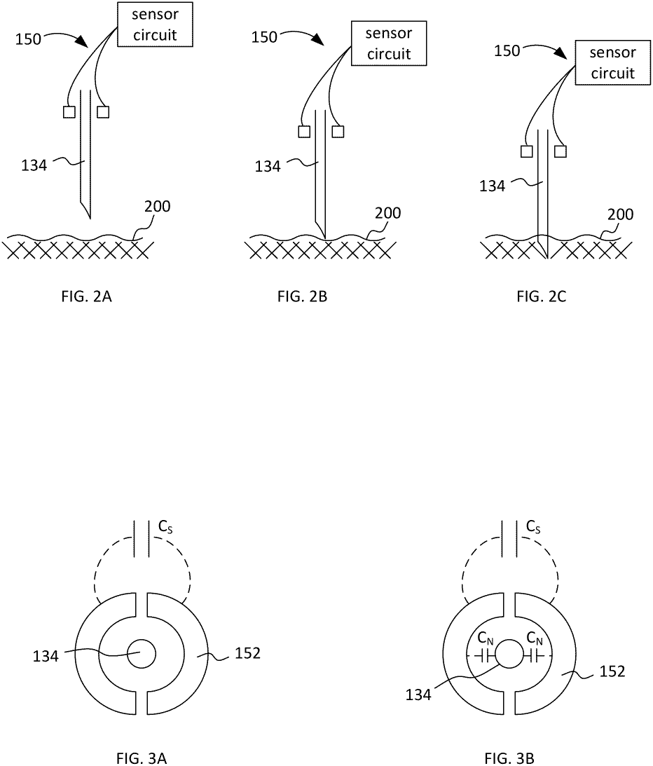

FIGS. 2A-C are schematic illustrations showing movement of a needle during insertion;

FIGS. 3A-B are schematic illustrations of how capacitive sensors work with a needle;

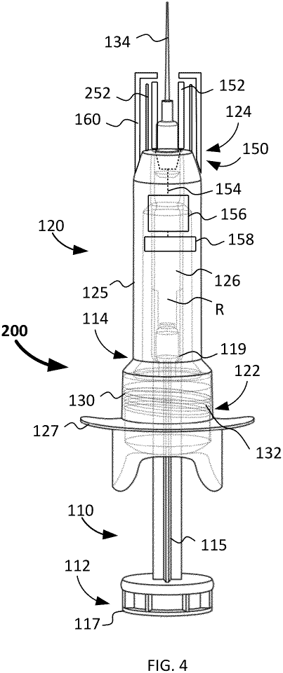

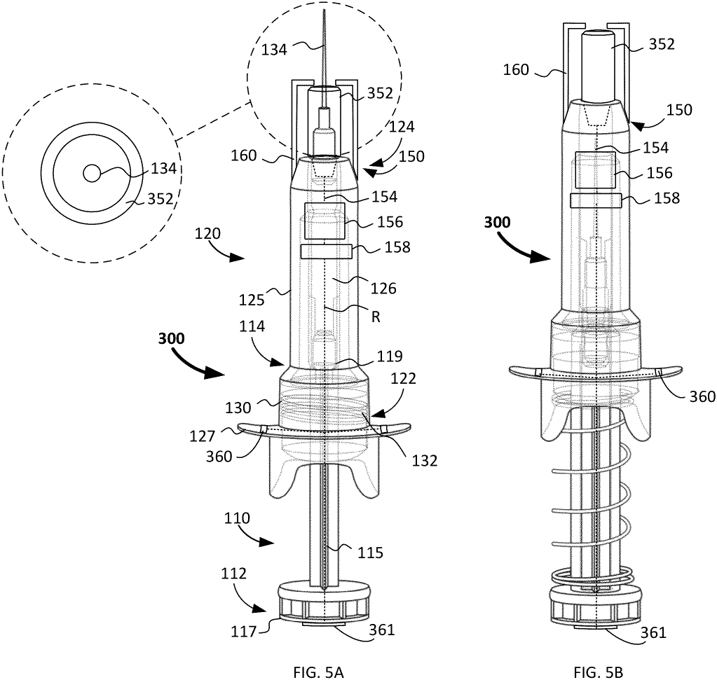

FIG. 4 is a schematic front view of one example of a pre-filled syringe having shielding electrodes;

FIGS. 5A-B are schematic front views of another embodiment of a pre-filled syringe having a ring-shaped electrode; and

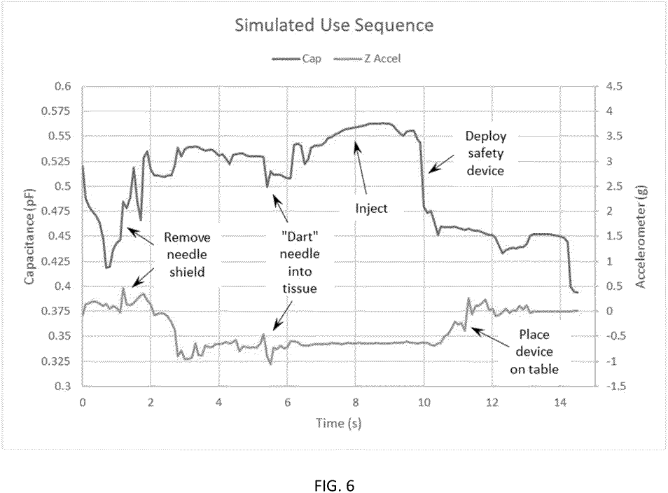

FIG. 6 is a diagram illustrating a capacitive needle sensor and accelerometer (Z-axis) output during a simulated use sequence.

Various embodiments will now be described with reference to the appended drawings. It is to be appreciated that these drawings depict only some embodiments of the disclosure and are therefore not to be considered limiting of its scope.

### DETAILED DESCRIPTION

Despite the various improvements that have been made to injectors and syringes, such as pre-filled syringes, conventional methods suffer from some shortcomings as discussed above.

Therefore, there is a need for further improvements to the devices and methods used to deliver medication and measure patient compliance. Among other advantages, the present disclosure may address one or more of these needs.

As used herein, the term “proximal,” when used in connection with a component of a syringe or injector, refers to the end of the component closest to the user’s hands when holding the device, whereas the term “distal,” when used in connection with a component of a syringe or injector, refers to the end of the component closest to the needle insertion site during use.

Likewise, the terms “trailing” and “leading” are to be taken as relative to the operator’s fingers (e.g., physician) of the syringe or injector. “Trailing” is to be understood as relatively close to the operator’s fingers, and “leading” is to be understood as relatively farther away from the operator’s fingers.

Reference is now made to FIGS. 1A-B, which show an exemplary prefilled-syringe 100 contained within a needle safety device having two states, a first state with the needle extended before injection (FIG. 1A), and a second state with the needle retracted within a barrel after the full injection has been completed (FIG. 1B). It will be understood that though a needle within a safety device is shown, the disclosure is not thus limited. For example, sensors of the present disclosure may be integrated into a specially-designed syringe barrel or through the use of a separate assembly that could be attached to the syringe prior to use. Additionally, though a pre-filled syringe with a staked needle is shown, it will be understood that the principles disclosed herein are equally applicable to other types of injectors (e.g., syringes with removable needles, auto-injectors, or on-body (wearable) injectors, etc.). Pre-filled syringe 100 generally comprises two main portions, a plunger rod 110 and a barrel 120. Plunger rod 110 generally extends between a proximal end 112 and a distal end 114, and comprises an elongated piston 115 extending between a plunger flange 117 and a coupler 119. In one embodiment, piston 115 has a cruciform cross-sectional shape.

A cylindrical barrel 120 extends between proximal end 122 and distal end 124 and comprises a body 125 defining a lumen 126 for accepting a portion of plunger rod 110. Body 125 further comprises a barrel flange 127 adjacent proximal end 122 and defines a reservoir “R” that holds a medicament, drug, saline, or other substance for injecting into a patient’s body. An internally threaded stopper 130 is disposed inside lumen 126 of body 125. In one embodiment, stopper 130 is made of an elastomeric material such as natural rubber, synthetic rubber, thermoplastic elastomers, or combinations thereof, and comprises an opening to receive and mate with coupler 119 of plunger rod 110 by advancing the plunger rod inside the barrel lumen 126 and rotating at least one of coupler 119 and stopper 130 relative to the other.

In this example, pre-filled syringe 100 includes a spring 132 operatively coupled to needle 134 to provide an additional safety mechanism. A cap 135 is also disposed over needle 134. Once cap 135 is removed, the user may pierce the patient’s skin with the needle, then push on plunger flange 117 to drive the plunger to deliver a medicament through needle 134 into the patient’s body. Spring 130 is configured so that, upon actuation and full delivery of the medicament, needle 134 will safely retract within barrel 120 and be locked inside to reduce the risk of needlestick injuries (FIG. 1B).

Syringe 100 of FIGS. 1A-B further illustrates the use of a capacitive sensor system to increase the safety of the device. Specifically, sensing system 150 may help to electronically detect that the needle of a syringe has been injected into human or animal tissue. In some examples, the detection system and corresponding method use a capacitance sensing approach in which the syringe’s needle (or cannula) is indirectly capacitively coupled (not in conductive contact) to a set of sensor electrodes and signal processing circuitry. Specifically, as shown in FIGS. 1A-B, a pair of conductive electrodes 152 are disposed on either side of needle 134 and spaced apart from the needle. Electrodes 152 may be made of a metal, such as copper, titanium, brass, silver, and platinum, or other suitable metals, alloys, conductive inks or polymers or materials. Electrodes 152 are shown as being two relatively flat plates, but it will be understood that the shape and/or size of the electrodes may be varied as desired. Electrodes 152 may be in electrical communication via wires 154 to a capacitance-to-digital converter (CDC) circuit 156 or similar suitable systems that employ a variety of detection methods including charge transfer (e.g., analog measuring techniques, analog signal converted to digital signal through the use of convention analog-to-digital converters, integrated circuits, etc.). Circuit 156, in turn, may be electrically coupled to a power source (not shown) and a microcontroller 158 having a processor and storage capability, for example, to store data or relay it to a computer or other suitable system. Optionally, housing of the body 125, when comprised of insulting material such as glass or plastic, may be extended distally to form an electrode guard 160 to surround the electrodes 152 to prevent them from making direct contact with the patient’s skin during administration, as this direct contact may result in short-circuiting of the electrodes or otherwise corrupting the capacitance signal rendering it unusable.

Sensing system 150 offers the primary advantage of not requiring direct physical contact between the electrodes and the needle. Because there is no physical contact between the electrodes 152 and needle 134, the system may be applied to a variety of syringe geometries for both staked and removable needles in glass or plastic syringes, either stand-alone or included with needle safety devices, while not interfering with the normal use or sterility of the syringe.

FIGS. 2A-C illustrate the use of sensing system 150 in various stages as needle 134 approaches the patient’s skin 200 (FIG. 2A), makes initial contact with the patient’s skin 200 (FIG. 2B), and pierces the patient’s skin (FIG. 2C). In these embodiments, it will be understood that the sensor circuit may obtain real-time capacitive measurements between the two electrodes and that the measured capacitance may change as the needle makes contact with the patient’s body, which has its own charge. To better illustrate this phenomenon, FIGS. 3A-B are shown in which arcuate-shaped electrodes 152 flank a needle 134. The two separated electrodes 152 are in close proximity to one another (e.g., between approximately 5 and 15 mm apart) and surround the needle to effectively form a capacitor with first capacitance Cs (FIG. 3A). Generally, capacitance, Cs, for a parallel plate electrode arrangement may be expressed mathematically as follows:

$${C}_{s} = \frac{{\text{ε}}_{r} {\text{ε}}_{0} A}{\text{d}} ,$$

where εᵣ is the permeability of the material between the two plates, ε₀ is the dielectric constant of free space, A is the area between the parallel plates, and d is the distance between the plates. In this equation, εᵣ will change based on the material between the plates (e.g., vacuum, dry air, etc.). In this instance, εᵣ will also be influenced by the presence of the needle when it contacts the patient’s body.

Thus, when a high-frequency signal (e.g., between 10 and 100 kHz) is applied to one electrode, it is coupled via the capacitor with capacitance Cs to the other electrode and detected by the sensor circuitry. Because it is partially within the electric field between the two capacitor electrodes, the needle would be capacitively coupled (not in contact) with the electrodes, shown as a parasitic capacitor C_{N}.

With the needle in air or pushed through the rubber septum of a vial, the parasitic capacitance C_{N} would be expected to be negligible, so the signal applied to one electrode would not be significantly influenced by the needle, and would be picked up on the other electrode through the net sensed capacitance Cs. Alternatively, when needle 134 is inserted into subcutaneous tissue, the value of the parasitic capacitance C_{N} would increase due to the relatively high electrical charge residing on the patient’s body, resulting in a net change of the overall capacitance Cs detected between the two electrodes 152. Net capacitance and changes in capacitance may be measured using conventional AC small signal techniques as well as using capacitance-to-digital converter (CDC) circuits or systems that employ a variety of detection methods including charge transfer.

The present invention comprises an injecting device comprising: a reservoir for containing a medicament; a needle in communication with the reservoir and configured to deliver the medicament to a patient’s body; at least two electrodes spaced apart from one another and disposed on opposing sides of the needle; and a capacitance-to-digital converter circuit configured to generate a signal and measure capacitance between the at least two electrodes. In another embodiment, the injection device comprises at least two electrodes that are flat. In another embodiment, the injection device comprises at least two electrodes that are arcuate.

In one embodiment, the capacitance sensor system may have the ability to detect very small capacitance changes, possibly in the range of several hundred femtoFarads (fF), as the system relies on sensing small differences in parasitic capacitance when the needle is in the air, or in the patient’s body. In some examples, the positions of the electrodes are close enough to the needle when the device is configured for injection such that a reasonably measurable capacitance signal can be obtained without interfering with the normal use of the device.

One application of the needle injection sensor concerns the prevention of intentional misuse of a prefilled syringe, also known as “dose splitting,” in which the contents of, typically, a prefilled syringe designed for subcutaneous injection is transferred into another container, such as a vial or intravenous bag, in preparation for off-label or otherwise unintended use. In some examples, the needle injection sensor system provides a signal that may be used in conjunction with an interlock mechanism to either prevent or allow the syringe plunger to be actuated, or to otherwise prevent or allow an injection from being administered, depending upon whether insertion into animal tissue has been detected. Thus, in one embodiment, if the system does not sense that the needle is being inserted into the patient’s skin, it may maintain the interlock mechanism to prevent dispensing the medicament for unintended use. Once insertion into tissue has been detected, the interlock may be released to allow the medicament to be dispensed. This information may be collected by the microcontroller and stored on the device. In some examples, the information may also be communicated to a computer or server to flag noncompliance. In some examples, the communicated information may also, alternatively or in addition, relate to the dose being properly delivered for adherence monitoring purposes.

Another application of the needle insertion sensor system includes the monitoring of treatment adherence in which the sensor, when coupled with a method for detecting that a predetermined volume of fluid was dispensed from a filled syringe, provides additional objective evidence that the injection was also administered into tissue. The additional capacitance detection allows the system to obtain robust information that detecting the dispensed volume alone cannot provide. That is, in this system it may be possible to know that the medication was dispensed fully from the syringe, and that it was inserted into tissue as opposed to a bag or vial or sink. This type of data may be useful, for example, when examining patient compliance during clinical trials.

The detection systems and methods described above may also be integrated into a prefilled syringe assembly, with or without a safety device, or it could be provided as an attachment to a conventional refillable syringe. Additionally, the output of the detection system may be combined with signals from other on-board motion sensors, such as an accelerometer or inertial measurement unit (IMU), to provide information about the use of the device for treatment adherence monitoring, to enhance the safety of use, or, when combined with a safety interlock, to disable intentional misuse.

Variations of the sensing system are possible. FIG. 4 illustrates one such variation. Syringe 200 is similar to syringe 100 and includes the same components with the exception of the addition of one or more secondary capacitive electrode 252 arranged with respect to the primary electrodes 152 to shield the primary electrodes 152 from the effects on the capacitance signal due to proximity of the hands or other body parts of the injection administrator and patient. In this example, the secondary electrodes 252 enable a differential measurement that compensates for the common-mode changes in capacitance signal baseline as the device is handled prior to injection.

FIGS. 5A-5B illustrates another alternative embodiment in which a syringe 300 utilizes a ring electrode 352 in proximity to (preferably completely encircling) the base of needle 134, to form one electrode of the sensor capacitor Cs. The other electrode of capacitance Cs may be formed in at least two different ways. First, the second electrode may be formed by making conductive contact to the needle itself, such as with a spring-loaded contact, such that the needle serves as the second electrode. Second, a conductive connection to the patient, such as with an adhesive patch electrode that would be connected to the sensor circuitry.

In a particular embodiment, the injection device comprises at least two electrodes comprising an electrically conductive material. In a further embodiment, the at least two electrodes are spaced from the needle by between 2 and 7 mm. In another embodiment, the at least two electrodes are not in physical contact with the needle. In another embodiment, the at least two electrodes are spaced from each other by between 5 and 15 mm. In another embodiment, the injection device comprises a pair of shielding electrodes disposed about the at least two electrodes. In a further embodiment, the shielding electrodes comprise an electrically conductive material.

In some examples, one possible method for connecting the person administering the injection to the sensing circuit is to use first insulated conductive pads 360 on either the underside (finger side) of the barrel flange 127 of the syringe assembly or a central conductive pad 361 on the plunger flange 117. Conductive pads 360 or central conductive pad 361 may be electrically connected with the capacitance-to-digital converter. FIGS. 5A-B illustrate both of those possibilities, but it will be understood that a syringe may include insulated conductive pads in either or both locations. When a second person is administering the injection, proximity of the two bodies or direct touching would serve to connect the administrator’s body to the patient’s body, effectively forming a larger reference electrode. The capacitance sensor circuit used for this embodiment may be the same type employed in the previously described embodiments. In at least some examples, due to large expected variations in the reference electrode portion of the sensed capacitance in this alternate embodiment, the capacitance measurement approach be capable of operating over a wider dynamic range and have the ability to establish a baseline reading prior to injection. For example, the system may include a smart detection algorithm that is capable of differentiating between different events of interest, possibly by combining the capacitance signal with a signal from at least one auxiliary sensor (e.g., accelerometer or inertial measurement unit).

In one embodiment, the injection device further comprises a barrel for containing the reservoir, the barrel having at least one barrel flange, and a plunger at least partially disposed within the barrel and translatable relative thereto to drive the medicament from the reservoir to the needle, the at least one barrel flange having an insulated conductive pad in electrical communication with the capacitance-to-digital converter circuit.

FIG. 6 illustrates one such example, in which a capacitive needle sensor and an accelerometer measuring along a z-axis are used in a simulated use sequence. The upper blue line shows the capacitance, while the lower red line shows the accelerometer g-force. As shown in this example, the two sensors may continuously measure their respective parameter and various landmarks may be identified in each of the two signals. From these two signals, the device may infer certain aspects of device use, such as when the device is being handled, the device orientation or vibration associated with administration of the injection, in order to further qualify the changes in capacitance as being associated with intended use. In some examples, this information may also be communicated for compliance monitoring to a computer or server. In some examples, a baseline reading prior to injection may be established via a “taring” operation in which the capacitance seen when the device is first handled (the handling being detected by an accelerometer, for example) is assumed to be “zero” and further changes are based on that value. The baseline value may vary with each use of a device or a baseline capacitance may be determined by the fixed electrode/needle configuration and would be relatively constant from unit to unit.

The interlock mechanism described above may be embodied in a number of forms including plunger locking pins or a ratchet pawl that is released by energizing an electromechanical, electromagnetic or memory metal (Nitinol) actuator. Alternatively, energizing a small heating element may change the state of a wax or adhesive that holds a spring-loaded pin or pawl in a position for preventing actuation of the syringe plunger thus releasing the plunger to be actuated. In some other examples, energizing a small heating element that changes the state of a solid material within the cannula may be used to block the flow of a medicament (e.g., a liquid).

In some examples, methods used for measuring dispensed volume for adherence monitoring purposes may include determination of the linear position of the syringe plunger through optical, mechanical or electronic means as well as determining the binary state of the deployment or non-deployment of a mechanical syringe safety device, which can only occur upon completion of a successful injection with a safety device-equipped prefilled syringe assembly.

In a particular embodiment, the present invention comprises an injecting device comprising a reservoir for containing a medicament, a needle in communication with the reservoir and configured to deliver the medicament to a patient’s body, a first ring-shaped electrode disposed about the needle and spaced away therefrom, a second reference electrode spaced away from the first ring-shaped electrode, and a capacitance-to-digital converted circuit configured to generate a signal and measure capacitance between the first ring-shaped electrode and the second reference electrode. In a further embodiment, the reference electrode is disposed bilaterally on a barrel flange. In yet another embodiment, the reference electrode is disposed on a plunger flange.

In a particular embodiment, the present invention comprises a method of administering a medicament comprising providing an injecting device including a reservoir for containing a medicament, a needle in communication with the reservoir and configured to deliver the medicament to a patient’s body, at least two electrodes spaced apart from one another and disposed on opposing sides of the needle, and a capacitance-to-digital converter circuit configured to generate a signal and measure capacitance between the at least two electrodes; measuring a first baseline capacitance between the at least two electrodes when the needle is not in physical contact with the patient’s body; and measuring a second capacitance between the two electrodes when the needle is in physical contact with the patient’s body; and determining if the needle was inserted in the patient’s body based on a difference between the first baseline capacitance and the second capacitance. In a further embodiment, the present invention comprises measuring a second capacitance comprises continuously measuring the second capacitance and comparing it to the first baseline capacitance until the difference between the first capacitance and the second capacitance above a predetermined threshold. In another embodiment, the present invention comprises storing the first baseline capacitance and the second capacitance on a microcontroller. In yet a further embodiment, the method of administering a medicament further comprises sending information to a third party that the needle has made physical contact with the patient’s body. In yet another embodiment, the method of administering a medicament further comprises maintaining an interlock device to prevent medicament from being expelled from the injecting device until the needle has made physical contact with the patient’s body. In another embodiment, the method of administering a medicament further comprises gathering auxiliary information from at least one auxiliary sensor and using the auxiliary information and the first baseline capacitance to infer a condition of the injecting device.

It is to be understood that the embodiments described herein are merely illustrative of the principles and applications of the present disclosure. For example, the number, positioning and arrangement of electrodes of the capacitance sensor may be varied. Moreover, certain components are optional, and the disclosure contemplates various configurations and combinations of the elements disclosed herein. It is therefore to be understood that numerous modifications may be made to the illustrative embodiments and that other arrangements may be devised without departing from the spirit and scope of the present disclosure as defined by the appended claims.

It will be appreciated that the various dependent claims and the features set forth therein can be combined in different ways than presented in the initial claims. It will also be appreciated that the features described in connection with individual embodiments may be shared with others of the described embodiments.

1. An injection device comprising:
a reservoir for containing a medicament;
a needle in communication with the reservoir and configured to deliver the medicament to a patient’s body;
at least two electrodes spaced apart from one another and disposed on opposing sides of the needle; and
a capacitance-to-digital converter circuit configured to generate a signal and measure capacitance between the at least two electrodes.

2. The injection device of claim 1, wherein the at least two electrodes are flat.

3. The injection device of claim 1, wherein the at least two electrodes are arcuate.

4. The injection device of claim 1, wherein the at least two electrodes comprise an electrically conductive material.

5. The injection device of claim 1, wherein the at least two electrodes are spaced from the needle by between 2 and 7 mm.

6. The injection device of claim 1, wherein the at least two electrodes are not in physical contact with the needle.

7. The injection device of claim 1, wherein the at least two electrodes are spaced from each other by between 5 and 15 mm.

8. The injection device of claim 1, further comprising a pair of shielding electrodes disposed about the at least two electrodes.

9. The injection device of claim 8, wherein the shielding electrodes comprise an electrically conductive material.

10. The injection device of claim 1, further comprising a barrel for containing the reservoir, the barrel having at least one barrel flange, and a plunger at least partially disposed within the barrel and translatable relative thereto to drive the medicament from the reservoir to the needle, the at least one barrel flange having an insulated conductive pad in electrical communication with the capacitance-to-digital converter circuit.

11. The injection device of claim 1, further comprising a barrel for containing the reservoir, and a plunger at least partially disposed within the barrel, the plunger having a plunger flange and being translatable relative to the barrel to drive the medicament from the reservoir to the needle, the plunger flange having a central conductive pad in electrical communication with the capacitance-to-digital converter circuit.

12. An injecting device comprising:
a reservoir for containing a medicament;
a needle in communication with the reservoir and configured to deliver the medicament to a patient’s body;
a first ring-shaped electrode disposed about the needle and spaced away therefrom;
a second reference electrode spaced away from the first ring-shaped electrode; and
a capacitance-to-digital converter circuit configured to generate a signal and measure capacitance between the first ring-shaped electrode and the second reference electrode.

13. The injecting device of claim 12, wherein the reference electrode is disposed bilaterally on a barrel flange.

14. The injecting device of claim 12, wherein the reference electrode is disposed on a plunger flange.

15. A method of administering a medicament, comprising:
providing an injecting device including a reservoir for containing a medicament, a needle in communication with the reservoir and configured to deliver the medicament to a patient’s body, at least two electrodes spaced apart from one another and disposed on opposing sides of the needle, and a capacitance-to-digital converter circuit configured to generate a signal and measure capacitance between the at least two electrodes;
measuring a first baseline capacitance between the at least two electrodes when the needle is not in physical contact with the patient’s body; and
measuring a second capacitance between the two electrodes when the needle is in physical contact with the patient’s body; and
determining if the needle was inserted in the patient’s body based on a difference between the first baseline capacitance and the second capacitance.

16. The method of claim 15, wherein measuring a second capacitance comprises continuously measuring the second capacitance and comparing it to the first baseline capacitance until the difference between the first capacitance and the second capacitance above a predetermined threshold.

17. The method of claim 15, further comprising storing the first baseline capacitance and the second capacitance on a microcontroller.

18. The method of claim 17, further comprising sending information to a third party that the needle has made physical contact with the patient’s body.

19. The method of claim 17, further comprising maintaining an interlock device to prevent medicament from being expelled from the injecting device until the needle has made physical contact with the patient’s body.

20. The method of claim 17, further comprising gathering auxiliary information from at least one auxiliary sensor and using the auxiliary information and the first baseline capacitance to infer a condition of the injecting device.
